(19) United States
(12) Patent Application Publication
(10) Pub. No.: US 2017/0184727 A1
(43) Pub. Date: Jun. 29, 2017

(54) HUMAN BODY RADIATION EXAMINING METHOD AND SYSTEM

(71) Applicant: Nuctech Company Limited, Beijing (CN)

(72) Inventors: Ziran Zhao, Beijing (CN); Zhiqiang Chen, Beijing (CN); Yuanjing Li, Beijing (CN); Wanlong Wu, Beijing (CN); Yingkang Jin, Beijing (CN); Chenguang Zhu, Beijing (CN)

(21) Appl. No.: 15/282,485

(22) Filed: Sep. 30, 2016

(30) Foreign Application Priority Data
Dec. 29, 2015 (CN) 201511008958.6
Dec. 29, 2015 (CN) 201521116652.8
Sep. 12, 2016 (CN) 201610818922.2

(51) Int. Cl. G01T 1/02; A61N 5/10

(52) U.S. Cl. CPC G01T 1/02; A61N 5/10

(57) ABSTRACT

A human body radiation examining method and system are disclosed. In one aspect, the human body radiation examining method comprises: identifying a person to be examined. The method further comprises retrieving an accumulative radiation dose of the person according to identification result. The method further comprises obtaining a predicted single radiation scanning dose of a human body radiation examining device intended to perform a current radiation examination. The method further comprises calculating a sum value of the accumulative radiation dose of the person and the predicted single radiation scanning dose of the human body radiation examining device. The method further comprises determining whether to perform the current radiation examination on the person according to whether the sum value exceeds a dose limit. In some embodiments, the human body radiation examining system and method can improve the security of the human body radiation examination.

### CROSS-REFERENCE TO RELATED APPLICATIONS

This application claims priority to Chinese Patent Application No. CN201610818922.2, filed on Sep. 12, 2016, Chinese Patent Application No. CN 201511008958.6 filed on Dec. 29, 2015, and Chinese Patent Application No. CN201521116652.8 filed on Dec. 29, 2015, incorporated herein by reference in their entity.

### BACKGROUND

Field

The disclosed technology relates to a human body radiation examining method and a human body radiation examining system for used in the field of security inspection, such as in an airport, a public transit station, customs, and the like, and/or other fields of radiation use on the human body for medical treatment such as in a hospital setting.

Description of the Related Technology

Human body radiation security inspection is used in airports, customs, a public transit station and other places. Common radiation examining techniques include a radiation perspective imaging technique and a radiation back-scattered imaging technique. The radiation perspective imaging technique transmits radiation in the direction of a human body to create a human body perspective image. The image is then analyzed and processed. The radiation perspective imaging technique can detect objects hidden within the human body. The radiation back-scattered technique uses a trace amount of radiation to scan the human body. The radiation is reflected back from the surface of the human body, and this reflected radiation can be used to obtain a human body surface profile image. This technique can detect dangerous goods hidden within the human body.

A personal radiation dose limit refers to an upper limit of a radiation dose permitted for an individual or a lower limit of a radiation dose unpermitted for the individual. Different radiation guarding systems stipulate corresponding dose limits. The dose limit involves a single radiation dose limit and an annual accumulative radiation dose limit. There is a need for a human body radiation examining device that satisfies requirements for the dose limit so as to ensure the safety of a human body.

Existing human body radiation examining devices simply consider a single radiation dose to a person to satisfy the requirement for radiation guarding. However, existing human body radiation examining devices do not directly measure and control an accumulative radiation dose to a person in a predetermined period. As people, living in modern society, are exposed to more radiation inspections, there is a substantial need for a system that accounts for the influence of accumulative radiation doses on human health.

### SUMMARY OF CERTAIN INVENTIVE ASPECTS

The disclosed technology includes a human body radiation examining system and a human body radiation examining method that are much safer to a person to be examined, and which can monitor and manage a single radiation dose of a person and an accumulative radiation dose of the person in different human body radiation examining devices in a predetermined period so as to avoid an excessive radiation dose applied to a person and cause a health hazard.

The disclosed technology includes a human body radiation examining method. The method includes identifying a person to be examined. The method further includes retrieving an accumulative radiation dose of the person according to identification result. The method further includes obtaining a predicted single radiation scanning dose of a human body radiation examining device intended to perform a current radiation examination. The method further includes calculating a sum value of the accumulative radiation dose of the person and the predicted single radiation scanning dose of the human body radiation examining device. The method further includes determining whether to perform the current radiation examination on the person according to a judgment whether the sum value exceeds a dose limit.

In an embodiment, the disclosed technology includes a human body radiation examining system. The system includes at least one human body radiation examining device configured to examine a human body with a radiation. The system further includes a personal identification device configured to identify an identity information of a person and send the identity information to the at least one human body radiation examining device. The system further includes a cloud server configured to store a single radiation dose information of the person during each radiation examination in one year and an annual accumulative radiation dose information; and a data processor configured to obtain a rated single radiation dose from the at least one human body radiation examining device and an annual accumulative radiation dose of the person from the cloud server and calculate a sum value of the rated single radiation dose and the annual accumulative radiation dose to determine whether to perform a current radiation examination on the person according to a judgment whether the sum value exceeds a dose limit.

The disclosed technology includes a human body radiation examining method and the human body radiation examining system. The human body radiation examining method and the human body radiation examining system includes methods and systems that can monitor and manage the single radiation dose, an accumulative radiation dose and an annual accumulative radiation dose of a person so as to ensure the radiation dose received by the person will not exceed the dose limit, thereby preventing radiation accidents.

In some embodiments, the current actual radiation dose of the person is converted at real time according to the average single pixel intensity value in the residual pixel region, or is measured at real time by using the personal radiation dosimeter. In some embodiments, it is possible to accurately obtain the actual current radiation dose of the person, thereby further improving the security of the human body radiation examining device.

Other objects and advantages of the disclosed technology will be apparent by the following description of embodiments with reference to the accompanying drawings.

### BRIEF DESCRIPTION OF THE DRAWINGS

FIG. 1 is a schematic view of a human body radiation examining system according to an embodiment of the disclosed technology;

FIG. 2 is a block diagram of a basic construction of a human body radiation examining device according to an embodiment of the disclosed technology;

FIG. 3 is a schematic view of a radiation scanning image of a person being examined according to an embodiment of the disclosed technology; and

FIG. 4 is a flow chart of a method for examining a human body using the human body radiation examining system as shown in FIG. 1 according to an embodiment of the disclosed technology.

### DETAILED DESCRIPTION OF CERTAIN ILLUSTRATIVE EMBODIMENTS

Embodiments of the disclosed technology will be clearly and completely described hereinafter with reference the accompanying drawings in exemplary embodiments of the disclosed technology. Obviously, the described embodiments are merely part of the embodiments of the disclosed technology, rather than all of the embodiments of the disclosed technology. Based on the embodiments of the disclosed technology, all other embodiments made by those skilled in the art without any inventive step will fall within the scope of the disclosed technology.

In addition, in the below detail description, for easy to explain, many specific details are set forth to provide a complete understanding to the disclosed technology. However, one or more embodiments can be obviously carried out without these specific details. In other cases, well-known structures and devices are illustrated to simplify the drawings.

In the following detail description, a number of specific details are set forth to provide a complete understanding to embodiments of the disclosed technology. However, one or more embodiments can be obviously carried out without these specific details. In other cases, well-known structures and devices are illustrated to simplify the drawings. Further, in all accompanying drawings, the same reference numeral is used to denote the same component or part.

FIG. 1 is a schematic view of a human body radiation examining system according to an exemplary embodiment of the disclosed technology. As shown in FIG. 1, the human body radiation examining system generally includes a personal identification device 11 and a human body radiation examining device 12 at a user end 10 and a cloud server 20 at a network end (cloud end), such as a dose information server.

The personal identification device 11 registers or identifies identity information of a person to be examined and sends the identity information to the human body radiation examining device 12. The personal identification device 11 includes an identification card reader, a fingerprint identification unit, a 2-dimensional bar code scanning gun, a M1 card reader and the like, for example.

The human body radiation examining device 12 performs a safety or medical examination on a human body with radiations. The human body radiation examining device 12 can interact and communicate with the cloud server 20, and transmit the identity information of the person identified by the personal identification device 11 to the cloud server 20 and receive annual accumulative radiation dose information of the person from the cloud server 20.

The cloud server 20 can communicate with the human body radiation examining device 12 to receive data of a single radiation dose of the person being examined each time, and automatically accumulate the data to obtain data of an accumulative radiation dose of the person such as a value of an annual accumulative radiation dose of the person and store the relevant data into the cloud server 20. The cloud server 20 may further store information such as a personal ID, an ID of a radiation scanning device, a scanning time, a single dose, an annual accumulative dose, an annual accumulative dose limit.

Although FIG. 1 schematically shows one human body radiation examining device 12, according to other embodiments of the disclosed technology, the human body radiation examining device 12 may include a plurality of human body radiation examining devices, each of which communicates with the cloud server 20 so as to form a dose monitoring network. The cloud server 20 serves to manage all the human body radiation examining devices 12 in the network. This network may be large or small. The smallest network may only include one human body radiation examining device. For example, in a case where the human body radiation examining device 12 is applied in a prison, there may be only one human body radiation examining device 12 to perform examination. Further, the dose limit stored in the cloud server 20 may be variably assigned and particularly determined according to local laws and regulations, industrial standards and the like regarding radiation protection. In some occasions, different dose limits may be applied to different persons according to gender, age or the like. All these information can be stored in a cloud server 20 for use.

When the plurality of human body radiation examining devices 12 are networked, each of the plurality of human body radiation examining devices 12 is communicated with the cloud server 20 to transmit data of a single radiation dose of the person at each human body radiation examining device 12 during each examination to the cloud server 20 so as to establish a database including information of each single radiation dose of the person during each examination in one year and an annual accumulative radiation dose of the person. The plurality of human body radiation examining devices may be of the same or different type. Each of the human body radiation examining devices may have different rated single radiation scanning doses based on different parameter settings.

The plurality of human body radiation examining devices 12 may be remotely or proximally communicated with the cloud server 20. A network connection between the plurality of human body radiation examining devices 12 and the cloud server 20 may be realized in a wired or wireless manner. The network connection may be a local network formed by several apparatuses. It may also be a cross-regional public network. Therefore, the human body radiation examining system of the disclosed technology may realize information sharing and co-management of a plurality of apparatuses so as to maximally secure the safety of a personal during radiation examination or inspection.

In addition, the human body radiation examining system according to the embodiments as described above further includes a data processor 30. FIG. 1 shows an example in which the data processor 30 is integrated into the cloud server 20. The data processor 30 is configured to obtain a rated single radiation scanning dose from the at least one of the human body radiation examining devices 12 and an annual accumulative radiation dose of the person from the cloud server 20 and calculate a sum value of the two doses to determine whether to perform a radiation examination on the person according to a judgment whether the sum value exceeds a dose limit.

Specifically, if the sum value of the annual accumulative radiation dose of the person and the rated single radiation scanning dose of the human body radiation examining device 12 exceeds the dose limit, it is determined not to perform the current radiation examination on the person; and if the sum value of the annual accumulative radiation dose of the person and the rated single radiation scanning dose of the human body radiation examining device 12 does not exceed the dose limit, it is determined to perform the current radiation examination on the person.

In the example shown in FIG. 1, the data processor 30 is integrated into the cloud server 20. In this case, when performing an examination on a human body of a person, the cloud server 20 may obtain personal identity information of the person to be examined and a rated single radiation scanning dose from the current human body radiation examining device 12 to perform the current examination. Then, the data processor 30 will add the rated single radiation scanning dose of the current human body radiation examining device 12 and an annual accumulative radiation dose of the person to obtain a sum value at the cloud server 20. Thereafter, it is determined whether to perform the current radiation examination on the person according to the judgment whether the sum value exceeds the dose limit set by the cloud server 20, and the determination result is then sent to the human body radiation examining device 12.

According to other embodiments of the disclosed technology, when there are a plurality of human body radiation examining devices, a data processor 30 may also be integrated into each human body radiation examining device 12. In this case, when performing an radiation examination on a human body of a person, the current human body radiation examining device 12 performing the current examination may transmit the personal identity information of the person identified by the personal identification device 11 to the cloud server 20 and obtain a value of an annual accumulative radiation dose of the person from the cloud server 20. Then, the date processor integrated into the human body radiation examining device 12 performing the current examination will add a rated single radiation scanning dose of the current human body radiation examining device 12 and the annual accumulative radiation dose of the person to obtain a sum value of the doses. Thereafter, it is determined whether to perform the current radiation examination on the person according to a judgment whether the sum value exceeds the dose limit set by the cloud server 20 or not.

As an example, the personal identification device 11 may be a device independent of the human body radiation examining device 12. For example, the personal identification device 11 may be separately placed on a console or held in hand by an operator. Alternatively, the personal identification device 11 may also be integrated into each human body radiation examining device 12, for example, fixed on a surface of the human body radiation examining device 12.

When the human body radiation examining system includes a single human body radiation examining device 12, the cloud server 20 may be placed in a server machine room, or it may also be integrated into the single human body radiation examining device 12. Moreover, the personal identification device 11 and the data processor 30 may also be integrated into the single human body radiation examining device 12.

If there are a plurality of human body radiation examining devices 12, the cloud server 20 may be placed in a machine room, or it may also be integrated into one of the plurality of human body radiation examining devices 12 networked with one another.

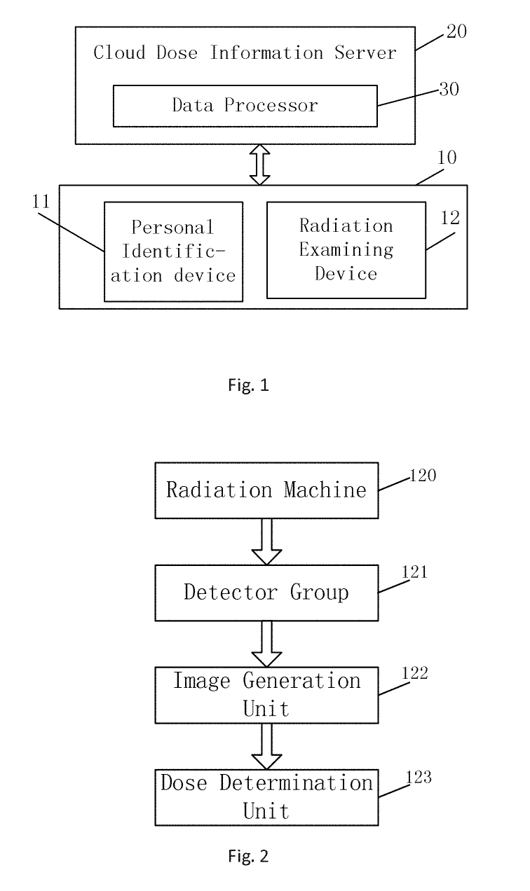

As shown in FIG. 2, each human body radiation examining device 12 may include: a radiation generator or a radiation source 120 for emitting radiations; a detector set 121 configured to receive the radiations and generate electrical signals, the detector set 121 may include of a plurality of detectors arranged in an array; an image generation unit 122 configured to convert the electrical signals of the detector set 121 into a radiation scanning image; and a dose determination unit 123 configured to extract intensity values of respective pixels in a residual pixel region expect an human body image in the radiation scanning image and calculate an average value of the intensity values of the respective pixels in the residual pixel region as an average single pixel intensity value so as to determine the current radiation dose of the person according to the average single pixel intensity value.

It will be apparent to those skilled in the art that the human body radiation examining device 12 may further include a mechanical drive unit, an electrical-control unit, a storage unit, a soft program and the like. Each human body radiation examining device 12 may have different output parameters, scanning speeds, human body radiation doses in a single scanning, conversion coefficients between the detector signals and the dose and the like, and the descriptions thereof in detail are omitted herein.

For each human body radiation examining device, a signal intensity of the detector thereof will reflect an amount of the dose of the radiation received by the human body. The signal intensity of the detector is reflected as pixel intensity (brightness) in the radiation scanning image. Therefore, for a human body radiation examining device 12 after setting-to-work test, the pixel intensity thereof is directly associated with the dose of the radiation. The higher the pixel intensity is, the larger the dose of the radiation is. For the same human body radiation examining device, the scanning dose thereof should be stable for a certain parameter setting. However, the radiation scanning dose will fluctuate under effect of environment, device condition and other factors during an actual radiation examination and thus be different from the rated radiation scanning dose. However, for each radiation examining device after calibration before putting into use, a conversion coefficient between the average single pixel intensity and the scanning dose without any object between the detector set 121 and the radiation generator is constant and stored in the human body radiation examining device. Therefore, it is possible to obtain an actual scanning dose value which reflects the current actual radiation dose of the person being examined through converting with the conversion coefficient based on the average single pixel intensity value in the scanning image obtained when there is no person or object between the detector set 121 and the radiation generator.

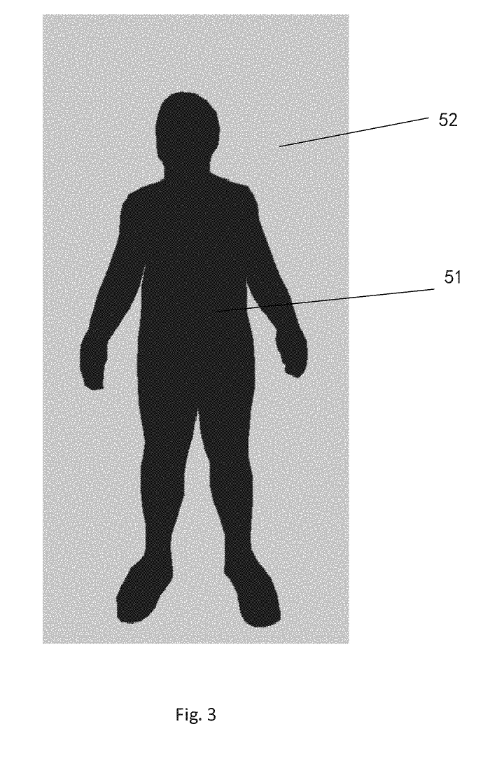

FIG. 3 is a schematic view of a radiation scanning image of a person being examined. As shown in FIG. 3, the radiation scanning image includes a human body image region 51 and a residual pixel region 52 except the human body image region 51. The human body image region 51 corresponds to a region in the detector set 121 in which the radiations are blocked by the human body (including also a region blocked by other object if there is other object). The residual pixel region 52 corresponds to a region in the detector set 121 in which the radiations are directly irradiated on the detectors without any obstacle. Because the residual pixel region 52 and the human body are synchronously irradiated with the radiations, it is possible to obtain a radiation dose value irradiated on a human body by measuring a single pixel intensity of the residual pixel region 52 and then converting the single pixel intensity into a corresponding radiation dose value according to a conversion coefficient of the human body radiation examining device 12 performing the current examination.

Further, since there may be errors in measurements of different detectors in the detector set, it is possible to calculate an average single pixel intensity value of the respective pixels in the residual pixel region 52 and then convert the average single pixel intensity value into the corresponding radiation dose. Because a large amount of residual pixels are used to perform an average calculation, the average value is very stable so as to correctly reflect a radiation output dose of the current scanning, thus accurately reflect the radiation dose received by the person being examined, thereby avoiding incorrect measurement of the radiation dose of the person due to a fluctuation in output of the human body radiation examining device 12 caused by various factors.

According to other embodiments of the disclosed technology, as shown in FIG. 1, the human body radiation examining system may further include a personal radiation dosimeter 40. The personal radiation dosimeter 40 is configured to be carried by the person being subjected to a radiation examination so as to directly measure a single radiation dose of the person. The personal radiation dosimeter 40 may be communicated with the human body radiation examining device 12 in the wired or wireless manner to send the measured single radiation dose to the human body radiation examining device 12. Alternatively, the personal radiation dosimeter 40 may be directly communicated with the cloud server 20 to directly upload the measured single radiation dose to the cloud server. Specifically, it is possible to place the personal radiation dosimeter 40 on the chest of the person being examined or at a position next to the person and flush with his/her chest. The personal radiation dosimeter 40 and the person are synchronously scanned with the radiations and the personal radiation dosimeter 40 directly measures the single radiation dose of the person.

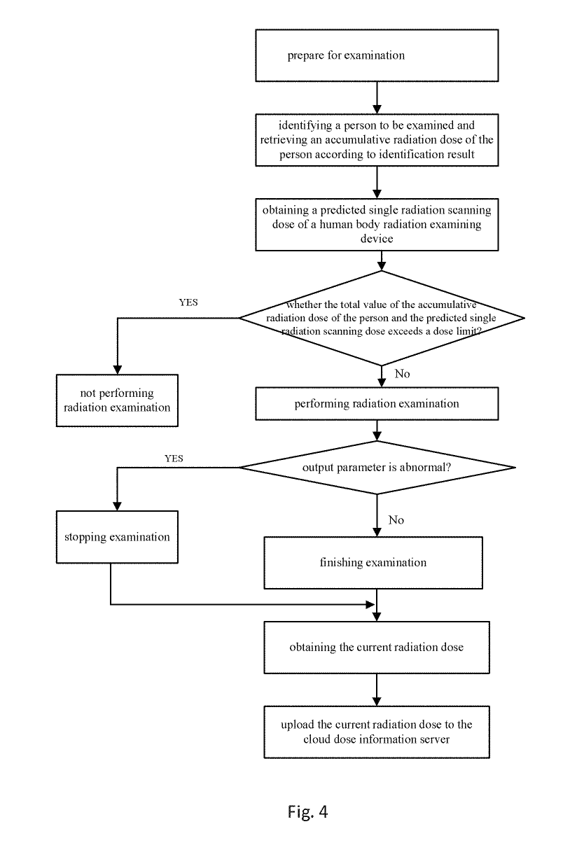

FIG. 4 is a flow chart of a method for examining a human body using the human body radiation examining system as shown in FIG. 1. As illustrated in FIG. 4, the method includes:
firstly registering or identifying a person when the person is ready to enter a scanning channel of the human body radiation examining device;
retrieving an accumulative radiation dose of the person, for example an annual accumulative radiation dose, according to a personal identification result;
obtaining a predicted single radiation scanning dose of the current radiation examination if the current radiation examination is performed based on a type of the human body radiation examining device, the predicated single radiation scanning dose may be a rated radiation scanning dose of the human body radiation examining device 12 performing the current radiation examination; and
adding the annual accumulative radiation dose of the person and the predicated single radiation scanning dose to obtain a sum value and determining whether to perform the current radiation examination on the person according to a judgment whether the sum value exceeds a dose limit.

Specifically, if the sum value exceeds a predetermined accumulative dose limit, it is determined not to perform the current radiation examination on the person so as to avoid the person from receiving excessive radiations and avoid radiation damage to health of the person. On the other hand, if the sum value obtained by adding the annual accumulative radiation dose of the person and the predicated single radiation scanning dose does not exceed the predetermined accumulative dose limit, it is determined to perform the current radiation examination on the person.

According to an example of the disclosed technology, the accumulative radiation dose may be an accumulated value of radiation doses which have been received by a person in one year. The dose limit may be an upper limit of accumulative radiation dose which may be received by a person in one year.

When it is determined to perform the current radiation examination on the person, it is possible to monitor an output parameter of the human body radiation examining device 12 at real time during performing the radiation examination on the person. If the output parameter is abnormal, the radiation examination is stopped. The radiation dose having been received by the person during the current partial radiation examination can be calculated by converting from a radiation dose in a complete radiation examination according to a ratio of a time period during which the current partial radiation examination is being performed to a time period during which a complete radiation examination is normally performed. The output parameter may for example be a source parameter such as a voltage, a current, a scanning speed of the radiation generator. If a source parameter exceeds its predetermined value ((abnormal)) due to a mis-operation of an operator or an equipment failure during the radiation scanning, the single radiation scanning dose may exceed the personal single radiation dose limit, which may cause health hazard to the human body. In this case, the human body radiation examining device 12 stops emitting the radiations to interrupt the scanning operation and calculates and records the current radiation dose having been received by the person being examined. Otherwise, the human body radiation examining device 12 will complete the current scanning and record the current radiation dose in a complete examination.

According to an exemplary embodiment of the disclosed technology, the above method further includes: after performing the radiation examination on the person, obtaining a current radiation dose of the person and transmitting the information of the current radiation dose to a cloud server 20 to update an annual accumulative value of radiation dose of the person.

Specifically, according to an exemplary embodiment of the disclosed technology, obtaining a current radiation dose of the person includes: obtaining a radiation scanning image of the person being examined currently; and processing the radiation scanning image to obtain the current radiation dose of the person based on an average single pixel intensity value of a residual pixel region 52 expect a human body image region 51 in the radiation scanning image by converting with a converting coefficient, which is stored in the human body radiation examining device 12 in advance. The average single pixel intensity value is an average of intensity values of respective pixels in the residual pixel region 52 expect the human body image region 51 in the radiation scanning image.

Since there may be errors in measurements of different detectors in the detector set, it is possible to calculate an average single pixel intensity value of the respective pixels in the residual pixel region 52 and then convert the average single pixel intensity value into the corresponding radiation dose. Because there are a large amount of residual pixels to be taken for an average calculation, the average value is very stable so as to correctly reflect a radiation output dose of the current scanning, thus accurately reflect the radiation dose of the person being scanned with radiations, thereby avoiding the radiation dose of the person from being incorrectly recorded due to a fluctuation in output dose of the human body radiation examining device 12 caused by various factors.

According to a simplified example of the embodiment of the disclosed technology, the radiation dose Y of the person during the current radiation examination may be calculated by the following formula:

Y=Y1·X/X1

wherein X is the average single pixel intensity value in the residual pixel region 52 in the radiation scanning image, Y1 is a value of the rated single radiation scanning dose of the human body radiation examining device 12 during the current human body radiation examination, X1 is a single pixel intensity value in a scanning image obtained with no person or object is scanned, corresponding to the rated single radiation scanning dose during the current human body radiation examination. X1 and Y1 may be pre-stored as equipment parameters in the human body radiation examining device.

According to another embodiment of the disclosed technology, obtaining a current radiation dose of the person being examined includes: obtaining the rated single radiation scanning dose of the human body radiation examining device 12 during the current human body radiation examination as the current radiation dose of the person.

According to a further embodiment of the disclosed technology, obtaining a current radiation dose of the person includes: the person carrying a personal radiation dosimeter 40 upon being examined, and the current radiation dose of the person is measured by the personal radiation dosimeter 40 at real time. For example, the personal radiation dosimeter 40 may be placed on the chest of the person or at a position next to the person and flush with his/her chest. The person being examined along with the personal radiation dosimeter 40 passes through an examining channel and the personal radiation dosimeter 40 communicates with the human body radiation examining device 12 in the wired or wireless manner. The personal radiation dosimeter 40 will accurately record the radiation dose of the person during each examination.

The methods for measuring and calculating the radiation dose of the person as described above may be used separately or in combination. When using the methods in combination, the personal dose should be calculated based on the maximal value so as to protect the health of the person.

Finally, the information of the current radiation dose of the person obtained by measuring and calculating as described above is uploaded to the cloud server to be accumulated with the personal accumulative radiation dose value stored in the cloud server 20 so as to update the accumulative radiation dose of the person.

The human body radiation examining device 12 and method according to the above embodiments of the disclosed technology, on one hand, can prevent the excessive single radiation dose from being generated due to the mis-operation or equipment failure. On the other hand, it is more important that the human body radiation examining device 12 and method can monitor an accumulative radiation dose and prevent the accumulative radiation dose from going beyond the accumulative radiation dose limit, thereby improving the security for human body radiation examination.

In addition, since the actual current radiation dose of a person being examined is converted at real time according to an average single pixel intensity value, or is measured at real time by using a personal radiation dosimeter, it is possible to accurately obtain the actual current radiation dose of the person, thereby further improving the security for human body radiation examination.

In the above embodiments of the disclosed technology, the annual accumulative radiation dose is an accumulative value of radiation doses having been received by a person in one year. The dose limit is an upper limit of an accumulative radiation dose, which is permitted to be received by a person in one year. The predicted single radiation scanning dose of the human body radiation examining device 12 is a rated single radiation scanning dose of the human body radiation examining device. However, the above embodiments are not restrictive. For example, the accumulative radiation scanning dose may be an accumulative value of radiation doses having received by a person in a quarter of a year. The dose limit may be an upper limit of an accumulative radiation dose, which is permitted to be received by a person in a quarter of a year. The predicted single radiation scanning dose of the human body radiation examining device 12 may be a dose value different from the rated single radiation scanning dose thereof. The human body radiation examining system of the disclosed technology may be applicable to various fields of human body radiation examination. The radiations may include for example X-rays, γ-rays and the like, which need to be monitored for health consideration.

Therefore, the above embodiments of the disclosed technology merely illustrate the principle and configuration thereof, rather than limiting the disclosed technology. It should be appreciated by those skilled in the art that any changes or modifications made to the embodiments of the disclosed technology will fall within the scope of the disclosed technology. The scope of the disclosed technology should be solely defined by the appended claims.

The previous description of the disclosed examples is provided to enable any person skilled in the art to make or use the present invention. Various modifications to these examples will be readily apparent to those skilled in the art, and the generic principles defined herein may be applied to other examples without departing from the spirit or scope of the invention. As will be recognized, certain embodiments of the inventions described herein can be embodied within a form that does not provide all of the features and benefits set forth herein, as some features can be used or practiced separately from others. The scope of certain inventions disclosed herein is indicated by the appended claims rather than by the foregoing description. All changes which come within the meaning and range of equivalency of the claims are to be embraced within their scope. Thus, the present invention is not intended to be limited to the examples shown herein but is to be accorded the widest scope consistent with the principles and novel features disclosed herein.

For purposes of summarizing the invention and the advantages achieved over the prior art, certain objects and advantages of the invention have been described herein above. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught or suggested herein without necessarily achieving other objects or advantages as may be taught or suggested herein.

All of these embodiments are intended to be within the scope of the invention herein disclosed. These and other embodiments will become readily apparent to those skilled in the art from the following detailed description of the preferred embodiments having reference to the attached figures, the invention not being limited to any particular preferred embodiment(s) disclosed.

What is claimed is:

1. A human body radiation examining method comprising:
identifying a person to be examined;
retrieving an accumulative radiation dose of the identified person;
obtaining a predicted single radiation scanning dose of a human body radiation examining device configured to perform a current radiation examination;
calculating a sum value of the accumulative radiation dose of the person and the predicted single radiation scanning dose of the human body radiation examining device; and
determining whether to perform the current radiation examination on the person according to whether the sum value exceeds a dose limit.

2. The human body radiation examining method according of claim 1, wherein the predicted single radiation scanning dose of the human body radiation examining device is a rated single radiation scanning dose of the human body radiation examining device.

3. The human body radiation examining method according of claim 1, wherein
the accumulative radiation dose is an accumulated value of radiation doses already received by the person in a year; and
the dose limit is an upper limit of an accumulated value of radiation doses permitted for the person in a year.

4. The human body radiation examining method according of claim 2, wherein determining whether to perform the current radiation examination on the person according to whether the sum value exceeds a dose limit comprises:
not performing the current radiation examination on the person if the sum value of the accumulative radiation dose of the person and the rated single radiation scanning dose of the human body radiation examining device exceeds the dose limit; and
performing the current radiation examination on the person if the sum value of the accumulative radiation dose of the person and the rated single radiation scanning dose of the human body radiation examining device does not exceed the dose limit.

5. The human body radiation examining method according of claim 4, further comprising:
monitoring an output parameter of the human body radiation examining device at real time during performing the radiation examination on the person and stopping the radiation examination if the output parameter is abnormal; and
calculating a radiation dose having been received by the person in the current partial radiation examination according to a ratio of a time period during which the current partial radiation examination is performed to a time period during which an entire radiation examination is to be performed.

6. The human body radiation examining method according of claim 4, further comprising:
after performing the radiation examination on the person, obtaining a current radiation dose of the person and transmitting a datum of the current radiation dose to a cloud server to update an annual accumulative value of radiation dose of the person.

7. The human body radiation examining method according of claim 6, wherein obtaining a current radiation dose of the person comprises:
obtaining a radiation scanning image of the person being examined currently; and
processing the radiation scanning image to obtain the current radiation dose of the person based on an average single pixel intensity value of a residual pixel region expect a human body image region in the radiation scanning image by converting with a converting coefficient, wherein the average single pixel intensity value is an average intensity value of respective pixels in the residual pixel region expect the human body image region in the radiation scanning image.

8. The human body radiation examining method according of claim 7, wherein the current radiation dose of the person is calculated by the following formula:
Y=Y1·X/X1
wherein X is the average single pixel intensity value in the residual pixel region in the current radiation scanning image, Y1 is a value of the rated single radiation scanning dose of the human body radiation examining device during the current human body radiation examination, X1 is a single pixel intensity value in a scanning image obtained with no person or object is scanned, corresponding to the rated single radiation scanning dose during the current human body radiation examination.

9. The human body radiation examining method according of claim 6, wherein obtaining a current radiation dose of the person comprises:
obtaining the rated single radiation scanning dose of the human body radiation examining device during the current human body radiation examination as the current radiation dose of the person.

10. The human body radiation examining method according of claim 6, wherein obtaining a current radiation dose of the person comprises:
the person carrying a personal radiation dosimeter upon being examined, and the current radiation dose of the person is measured by the personal radiation dosimeter at real time.

11. The human body radiation examining method according of claim 1, wherein the dose limit is variably assigned.

12. A human body radiation examining system comprising
at least one human body radiation examining device configured to examine a human body with a radiation;
a personal identification device configured to identify an identity information of a person and send the identity information to the at least one human body radiation examining device;
a cloud server configured to store a single radiation dose information of the person during each radiation examination in one year and an annual accumulative radiation dose information; and
a data processor configured to obtain a rated single radiation dose from the at least one human body radiation examining device and an annual accumulative radiation dose of the person from the cloud server and calculate a sum value of the rated single radiation dose and the annual accumulative radiation dose to determine whether to perform a current radiation examination on the person according to whether the sum value exceeds a dose limit.

13. The human body radiation examining system according of claim 12, further comprising a plurality of human body radiation examining devices each communicated with the cloud server to transmit data of the single radiation dose of the person at each human body radiation examining device during each radiation examination to the cloud server and accumulate the annual radiation dose so as to establish a data base including a personal radiation dose information for each radiation examination in one year and information of the annual accumulative radiation dose.

14. The human body radiation examining system according of claim 13, wherein the plurality of human body radiation examining devices are the same or different types of human body radiation examining device.

15. The human body radiation examining system according of claim 12, wherein the dose limit is stored in the cloud server, and the dose limit is variably assigned by the cloud server.

16. The human body radiation examining system according of claim 12, wherein the data processor is integrated into each human body radiation examining device or into the cloud server.

17. The human body radiation examining system according of claim 12, wherein the personal identification device is integrated into each human body radiation examining device.

18. The human body radiation examining system according of claim 12, further comprising a single human body radiation examining device, into which the personal identification device, the cloud server and the data processor are integrated.

19. The human body radiation examining system according of claim 12, further comprising a personal radiation dosimeter, the personal radiation dosimeter is carried by the person being examined for measuring the single radiation dose of the person.

20. The human body radiation examining system according of claim 12, wherein the human body radiation examining device comprises:
a radiation generator or a radiation source for emitting radiations;
a detector set configured to receive the radiations and generate electrical signals;
an image generation unit configured to convert the electrical signals of the detector set into a radiation scanning image; and
a dose determination unit configured to extract intensity values of respective pixels in a residual pixel region expect a human body image in the radiation scanning image and calculate an average value of the intensity values of the respective pixels in the residual pixel region as an average single pixel intensity value so as to obtain a current radiation dose of the person by converting with a concerting coefficient based on the average single pixel intensity value.
